SEQUENCE LISTING (32 sequences)

SEQ ID NO: 1 - protein, 15 aa, Artificial Sequence
  Synthetic peptide
AAAYAAAAAAKAAAA

SEQ ID NO: 2 - protein, 15 aa, Artificial Sequence
  Synthetic peptide
AEKYAAAAAAKAAAA

SEQ ID NO: 3 - protein, 15 aa, Artificial Sequence
  Synthetic peptide
AKEYAAAAAAKAAAA

SEQ ID NO: 4 - protein, 15 aa, Artificial Sequence
  Synthetic peptide
AKKYAAAAAAKAAAA

SEQ ID NO: 5 - protein, 15 aa, Artificial Sequence
  Synthetic peptide
AEAYAAAAAAKAAAA

SEQ ID NO: 6 - protein, 15 aa, Artificial Sequence
  Synthetic peptide
KEAYAAAAAAKAAAA

SEQ ID NO: 7 - protein, 15 aa, Artificial Sequence
  Synthetic peptide
AEEYAAAAAAKAAAA

SEQ ID NO: 8 - protein, 15 aa, Artificial Sequence
  Synthetic peptide
AAEYAAAAAAKAAAA

SEQ ID NO: 9 - protein, 15 aa, Artificial Sequence
  Synthetic peptide
EKAYAAAAAAKAAAA

SEQ ID NO: 10 - protein, 15 aa, Artificial Sequence
  Synthetic peptide
AAKYEAAAAAKAAAA

SEQ ID NO: 11 - protein, 15 aa, Artificial Sequence
  Synthetic peptide
AAKYAEAAAAKAAAA

SEQ ID NO: 12 - protein, 15 aa, Artificial Sequence
  Synthetic peptide
EAAYAAAAAAKAAAA

SEQ ID NO: 13 - protein, 15 aa, Artificial Sequence
  Synthetic peptide
EKKYAAAAAAKAAAA

SEQ ID NO: 14 - protein, 15 aa, Artificial Sequence
  Synthetic peptide
EAKYAAAAAAKAAAA

SEQ ID NO: 15 - protein, 15 aa, Artificial Sequence
  Synthetic peptide
AEKYAAAAAAAAAAA

SEQ ID NO: 16 - protein, 15 aa, Artificial Sequence
  Synthetic peptide
AKEYAAAAAAAAAAA

SEQ ID NO: 17 - protein, 15 aa, Artificial Sequence
  Synthetic peptide
AKKYEAAAAAAAAAA

SEQ ID NO: 18 - protein, 15 aa, Artificial Sequenece
  Synthetic peptide
AKKYAEAAAAAAAAA

SEQ ID NO: 19 - protein, 15 aa, Artificial Sequence
  Synthetic peptide
AEAYKAAAAAAAAAA

SEQ ID NO: 20 - protein, 15 aa, Artificial Sequence
  Synthetic peptide
KEAYAAAAAAAAAAA

SEQ ID NO: 21 - protein, 15 aa, Artificial Sequence
  Synthetic peptide
AEEYKAAAAAAAAAA

SEQ ID NO: 22 - protein, 15 aa, Artificial Sequence
  Synthetic peptide
AAEYKAAAAAAAAAA

SEQ ID NO: 23 - protein, 15 aa, Artificial Sequence
  Synthetic peptide
EKAYAAAAAAAAAAA

SEQ ID NO: 24 - protein, 15 aa, Artificial Sequence
  Synthetic peptide
AAKYEAAAAAAAAAA

SEQ ID NO: 25 - protein, 15 aa, Artificial Sequence
  Synthetic peptide
AAKYAEAAAAAAAAA

SEQ ID NO: 26 - protein, 15 aa, Artificial Sequence
  Synthetic peptide
EKKYAAAAAAAAAAA

SEQ ID NO: 27 - protein, 15 aa, Artificial Sequence
  Synthetic peptide
EAKYAAAAAAAAAAA

SEQ ID NO: 28 - protein, 15 aa, Artificial Sequence
  Synthetic peptide
AEYAKAAAAAAAAAA

SEQ ID NO: 29 - protein, 15 aa, Artificial Sequence
  Synthetic peptide
AEKAYAAAAAAAAAA

SEQ ID NO: 30 - protein, 15 aa, Artificial Sequence
  Synthetic peptide
EKYAAAAAAAAAAAA

SEQ ID NO: 31 - protein, 15 aa, Artificial Sequence
  Synthetic peptide
AYKAEAAAAAAAAAA

SEQ ID NO: 32 - protein, 15 aa, Artificial Sequence
  Synthetic peptide
AKYAEAAAAAAAAAA

(19) United States
(12) Patent Application Publication
(10) Pub. No.: US 2009/0130121 A1
(43) Pub. Date: May 21, 2009

(54) Synthetic Peptide Copolymers for Treatment and Prevention of Cardiovascular Disorders

(75) Inventors: Ruth Arnon, Rehovot (IL); Arkady-Avi Kotlyar, Ness Ziona (IL); Aviv Shayish, Yechiel (IL); Dror Haratz, Ramat-Gan (IL)

Correspondence Address: COOPER & DUNHAM, LLP, 30 Rockefeller Plaza, 20th Floor, NEW YORK, NY, 10112, US

(73) Assignee: Yeda Research and Development Co., Ltd.

(21) Appl. No.: 11/883,734

(22) PCT Filed: Feb. 2, 2006
(86) PCT No.: PCT/IL06/00134

§ 371 Date: Dec. 30, 2008

Related U.S. Application Data
(60) Provisional application No. 60/648,708, filed on Feb. 2, 2005.

(51) Int. Cl. A61K 38/00; A61K 38/07; A61K 38/06; A61K 38/10; A61K 39/395; A61P 9/00

(52) U.S. Cl. 424/158.1; 514/2; 514/18; 514/14

(57) ABSTRACT

The present invention relates to the use of random or ordered copolymers including the known copolymer glatiramer (also known as Copolymer 1) and Copolymer 1-related heteropolymers or ordered peptides, for treating or preventing cardiovascular diseases and disorders.

### FIELD OF THE INVENTION

The present invention relates to the use of random or ordered copolymers including the known copolymer glatiramer (also known as Copolymer 1) and Copolymer 1-related heteropolymers or ordered peptides, for treating or preventing cardiovascular diseases and disorders.

### BACKGROUND OF THE INVENTION

### Cardiovascular Diseases and Disorders

Myocardial ischemic disorders occur when cardiac blood flow is restricted (ischemia) and/or when oxygen supply to the heart muscle is compromised (hypoxia) such that the heart's requirement for oxygen is not met by the supply. Coronary artery disease (CAD) arising from arteriosclerosis, particularly atherosclerosis, is the most common cause of ischemia.

Atherosclerosis is one of the main causes of death in the United States, Europe, and parts of Asia. Atherosclerosis has many features of a chronic inflammatory disease, and it has been shown in a large number of studies to be associated with immune system activation, particularly macrophages and T lymphocytes (Varadhachary et al., 2001, Cell Immunol. 213(1):45-51). The major class of T lymphocytes present in atherosclerotic lesions is CD4+, which may differentiate into Th1 or Th2 lineage, together with the Th1 inducer IL-12, on both the mRNA and protein levels (Varadhachary et al., ibid). The Th2 inducer IL-10 is also present, although in a far more limited mode (Varadhachary et al., ibid). There is also evidence for the role of Th1 lymphocytes as manifested by the detection of interferon-gamma (IFN-γ) mRNA and protein in atherosclerotic lesions (Zhou et al., 1998. J. Clin. Invest. 101:1717-1725).

Mice with a targeted disruption of the apoE gene develop profound atherosclerosis, including atherosclerotic lesions in both the aortic arch and the ascending aorta. These mice develop atherosclerotic lesions after about nine months on a normal diet or after only three months on a high fat diet, and are used as a model of cardiovascular disease. However compound knock-out mice that were deficient in apoE and also in IFN-γ exhibited a substantial reduction in atherosclerotic lesion size compared to the apoE knock-out mice (Gupta et al., 1997, J Clin Invest. 99:2752-2761). Injection of IFN-γ or the IFN-γ-releasing factors IL-12 and IL-18, into apoE deficient mice resulted in enhancement of the disease (Lee et al., 1999, Arterioscler Thromb Vasc Biol. 19:734-742). Decreasing the appearance of Th1 cells by antibodies (α-CD4⁺) or cytokines (IL-4) also caused a marked reduction in lesion size, suggesting that Th phenotype is indeed important in fatty lesion development (Huber et al., 2001, Circulation. 103:2610-2616).

Currently available approaches for treating atherosclerosis include the use of non-specific drugs for treating hypertension, such as calcium-channel blockers, beta-blockers, angiotensin-converting enzyme (ACE) inhibitors, angiotensin II receptor blockers, vasodilators, cardiac glycosides and diuretics, cholesterol-lowering drugs, and Apolipoprotein A-1 (Apo-A1) derivatives. However, these drugs are non-specific to atherosclerosis but rather are directed to alleviating symptoms associated with atherosclerosis, such as high blood pressure, rupture of vessel walls and blood clot formation.

### Copolymer 1

Copolymer 1 (Cop 1, also known by the trivial chemical name glatiramer acetate), a synthetic random copolymer composed of the four amino acids: L-Glu, L-Lys, L-Ala, and L-Tyr (hereinafter “Cop 1”), is approved for the treatment of multiple sclerosis under the name of COPAXONE® (Teitelbaum et al., in: Shoenfeld Y. Ed. The Decade in Autoimmunity. Elsevier 183, 1998). Copolymer 1 was demonstrated to have Th cell related immunomodulatory effects.

Cop 1 is a high molecular weight synthetic basic random copolymer consisting of L-Ala, L-Glu, L-Lys and L-Tyr residues in the molar ratio of about 6 parts Ala to 2 parts Glu to 4.5 parts Lys to 1 part Tyr, first described in U.S. Pat. No. 3,849,550 as an agent for treatment or prevention of experimental allergic encephalomyelitis (EAE), a disease resembling multiple sclerosis (MS) that can be induced in susceptible animals. D-Copolymer 1 or D-Cop 1, in which the four amino acids have the D-configuration, namely a random copolymer containing the D-Ala, D-Glu, D-Lys and D-Tyr residues, has also been described (Webb et al., 1976, Immunochemistry, 13:333-337).

Recently it was found that in animal models, Cop 1 provides a beneficial effect for several additional disorders. Cop 1 suppresses the immune rejection manifested in graft versus host disease (GVHD) in case of bone marrow transplantation, as disclosed in U.S. Pat. No. 5,858,964, and in graft rejection in case of solid organ transplantation as disclosed in WO 00/27417. WO 2005/009333 discloses compositions and methods for the prevention and treatment of graft rejection, comprising Copolymer 1 and Copolymer 1-related random heteropolymers in conjunction with immunosuppressive agents.

WO 01/52878 and WO 01/93893 disclose that Cop 1, Cop 1-related peptides and polypeptides as well as T cells activated therewith protect CNS cells from glutamate toxicity and prevent or inhibit neuronal degeneration or promote nerve regeneration in the central nervous system (CNS) and peripheral nervous system (PNS). Kipnis et al. describe the therapeutic effect of Cop 1 in neurodegenerative diseases, such as optic neuropathies and glaucoma (Kipnis et al., 2002, Trends Mol Med, 8(7):319-23).

Cop 1 and related copolymers and peptides have been disclosed in WO 00/05250 assigned to the common assignee of the present invention, hereby incorporated by reference in its entirety as if fully disclosed herein, for treating autoimmune diseases. Nowhere in the background art is it disclosed or suggested that Cop 1 and related copolymers may be suitable for treatment of cardiovascular diseases and disorders. There exists a long-felt need for specific means for preventing and for treating cardiovascular diseases and disorders, including atherosclerosis. The present invention satisfies this need and provides related advantages as well.

### SUMMARY OF THE INVENTION

The present invention provides methods for treating cardiovascular diseases and disorders using immunomodulators. Specifically, the methods of the present invention disclose for the first time random or ordered copolymers including Copolymer 1 and Copolymer 1-related heteropolymers or peptides, for treating cardiovascular diseases and disorders including, but not limited to, atherosclerosis and myocarditis.

The present invention is based in part on the surprising discovery that Copolymer 1 exhibits an unexpected inhibitory effect on the early development of atherosclerosis. While application of the currently used drugs for treating atherosclerosis is limited to treatment of the established disease and cannot be used to prevent the disease before its onset or at the early stages of its development, the activity of Cop 1 and Cop 1-related heteropolymers as described in the present invention inhibits the development of atherosclerosis at its early stage. Moreover, it is known in the art that the activity of Cop 1 and Cop 1-related heteropolymers or ordered peptides involves MHC blocking as well as Th1 to Th2 cytokine shift, wherein elevated levels of Th 1 cells are associated with the appearance and development of various cardiovascular diseases including atherosclerosis among others. Thus, without wishing to be bound by any particular theory or mechanism of action, Cop 1 may be used for preventing atherosclerosis as its activity does not depend on the symptoms of the disease but rather affects the relative amount or appearance of Th1 cells in the blood.

According to one aspect, the present invention provides a method for treating or preventing cardiovascular diseases and disorders in a subject in need thereof, comprising administering a therapeutically effective amount of at least one copolymer, the copolymer selected from copolymer 1 and a copolymer 1-related heteropolymer wherein said copolymer comprises at least three amino acids selected from at least three of the following groups:
(a) lysine and arginine;
(b) glutamic acid and aspartic acid;
(c) alanine, glycine and valine;
(d) tyrosine, tryptophan and phenylalanine.

According to one embodiment, the at least one copolymer is selected from the group consisting of a random copolymer, an ordered copolymer and an ordered peptide.

According to various embodiments of the present invention, the random or ordered copolymers and peptides to be used in the method of the present invention comprise a suitable quantity of amino acids having positive electrical charge, such as lysine or arginine, in combination with amino acids with a negative electrical charge (preferably in a lesser quantity), such as glutamic acid or aspartic acid, optionally in combination with electrically neutral amino acids such as alanine, glycine or valine, serving as a filler, and optionally with phenylalanine, tyrosine or tryptophan, the optional amino acids adapted to confer on the copolymer immunogenic properties.

According to another embodiment, the at least one copolymer contains four different amino acids each selected from one of groups (a) to (d). According to yet another embodiment, the at least one copolymer comprises alanine, glutamic acid, lysine and tyrosine, of net overall positive electrical charge having a molecular weight of about 2 to 40 kilodaltons. According to alternative embodiments the molecular weight is of about 13 to 18 kilodaltons.

According to a further embodiment, the at least one copolymer comprises alanine, glutamic acid, lysine, and tyrosine in the molar ratios of: glutamic acid about 0.14, alanine about 0.43, tyrosine about 0.10 and lysine about 0.33. According to a preferred embodiment, the molar ratios of the amino acid residues include the following relative molar ratios: 0.17 glutamic acid to 0.38 lysine to 0.49 alanine to 0.1 tyrosine. According to another preferred embodiment, said relative molar ratios are 0.19 glutamic acid to 0.4 lysine to 0.6 alanine to 0.1 tyrosine.

According to yet a further embodiment, the average molecular weight of the copolymer of the invention is about 2,000-40,000 Da. According to various embodiments, the average molecular weight of the copolymer of the invention is about 2,000-18,000 Da. According to certain embodiments, the average molecular weight of the copolymer of the invention is about 4,500-17,000 Da. According to some embodiments the copolymer is glatiramer acetate having an average molecular weight of about 5,000-9,000 Da. According to various embodiments, the copolymer is glatiramer acetate having an average molecular weight of about 6,000-8,000 Da. According to a preferred embodiment, the average molecular weight of the copolymer of the invention is about 13,000-1 8,000 Da. According to another preferred embodiment, the average molecular weight of the copolymer of the invention is about 15,000 Da.

It is clear that the foregoing embodiments are given by way of example only, and that the copolymer and the compositions comprising same may be varied both with respect to the constituents and relative proportions of the constituents if the above general criteria as defined in the first aspect are adhered to.

According to yet another embodiment, the at least one copolymer contains three different amino acids each one selected from groups (a) to (d). These copolymers may be random or ordered and are herein referred to as terpolymers. According to one embodiment, the at least one copolymer comprises random or ordered terpolymer consisting of three different amino acids, each selected from a different one of the following groups:
(a) lysine and arginine;
(b) glutamic acid and aspartic acid;
(c) phenylalanine, tyrosine and tryptophan.

According to another embodiment, said copolymer contains glutamic acid, tyrosine, and lysine, in the molar ratio of from about 0.005 to about 0.300 glutamic acid, from about 0.005 to about 0.250 tyrosine, and from about 0.3 to about 0.7 lysine, and a pharmaceutically acceptable carrier. This terpolymer, hereinafter designated YEK, is preferably substantially free of alanine.

In a further embodiment, the molar ratios of glutamic acid, tyrosine, and lysine are about 0.26 to about 0.16 to about 0.58, respectively. According to certain embodiments, the average molecular weight of YEK is about 2,000-40,000 Da. According to other embodiments. the average molecular weight of YEK is about 3,000-35,000 Da.

According to yet other embodiments, the average molecular weight of YEK is about 5,000-25,000 Da. According to a preferred embodiment, the average molecular weight of YEK is about 13,000-18,000 Da. According to another preferred embodiment, the average molecular weight of YEK is about 15,000 Da.

It is possible to substitute arginine for lysine, aspartic acid for glutamic acid or phenylalanine or tryptophan for tyrosine.

According to an alternative embodiment, the terpolymer consists of three different amino acids, each selected from a different one of the following groups:
(a) lysine and arginine;
(b) alanine, glycine and valine;
(c) phenylalanine, tyrosine or tryptophan.

According to this alternative embodiment, the at least one terpolymer contains tyrosine, alanine and lysine, in the molar ratio of from about 0.005 to about 0.25 tyrosine, from about 0.3 to about 0.6 alanine, and from about 0.1 to about 0.5 lysine, along with a pharmaceutically acceptable carrier. This terpolymer, hereinafter designated YAK, is preferably substantially free of glutamic acid.

According to this alternative embodiment, the molar ratios of tyrosine, alanine and lysine are about 0.10 to about 0.54 to about 0.35, respectively. According to certain embodiments, the average molecular weight of YAK is about 2,000-40,000 Da. According to other embodiments, the average molecular weight of YAK is about 3,000-35,000 Da. According to yet other embodiments, the average molecular weight of YAK is about 5,000-25,000 Da. According to a preferred embodiment, the average molecular weight of YAK is about 13,000-18,000 Da. According to another preferred embodiment, the average molecular weight of YAK is about 15,000 Da.

According to certain embodiments, it is possible to substitute arginine for lysine, glycine or valine for alanine or phenylalanine or tryptophan for tyrosine.

According to yet another alternative embodiment, the at least one copolymer contains a terpolymer consisting of three different amino acids, each selected from a different member of the following groups:
(a) glutamic acid and aspartic acid;
(b) alanine, glycine and valine;
(c) phenylalanine, tyrosine and tryptophan.

According to this alternative embodiment, the at least one copolymer comprises a terpolymer containing tyrosine, glutamic acid and alanine, in the molar ratio of from about 0.005 to about 0.25 tyrosine, from about 0.005 to about 0.3 glutamic acid, and from about 0.005 to about 0.8 alanine, and a pharmaceutically acceptable carrier. This terpolymer, hereinafter designated YEA, is preferably substantially free of lysine.

According to this alternative embodiment, the molar ratios of glutamic acid, alanine, and tyrosine are about 0.21 to about 0.65 to about 0.14, respectively. The average molecular weight of YEA is about 2,000-40,000. According to certain embodiments, the average molecular weight of YEA is about 3,000-35,000 Da. According to other embodiments, the average molecular weight of YEA is about 5.000-25.000 Da. According to a preferred embodiment, the average molecular weight of YEA is about 13,000-18,000 Da. According to another preferred embodiment, the average molecular weight of YEA is about 15,000 Da. It is possible to substitute aspartic acid for glutamic acid, glycine for alanine, and phenylalanine or tryptophan for tyrosine.

The at least one copolymers to be used in the method of the invention can be composed of L- or D-amino acids or mixtures thereof. As is known by those of skill in the art, L-amino acids occur in most natural proteins. However, D-amino acids are commercially available and can be substituted for some or all of the amino acids used to make the copolymers used in the present invention. The present invention contemplates the use of copolymers containing both D- and L-amino acids, as well as copolymers consisting essentially of either L- or D-amino acids.

According to various embodiments of the present invention, the at least one copolymer may be a random polypeptide from about 15 to about 100 amino acids. According to certain embodiments, the at least one copolymer may be a random polypeptide from about 40 to about 80 amino acids in length. According to various alternative embodiments, the at least one copolymer is an ordered synthetic peptide of from 6 to 25 amino acids. According to various alternative embodiments, the at least one copolymer is an ordered synthetic peptide of 10 to 20 amino acids. According to yet other embodiments, oligomeric forms of the peptides may be used in the method of the invention, having from about 15 to about 100 amino acids, alternatively from about 40 to about 80 amino acids in length.

According to various embodiments, the at least one ordered synthetic peptide is selected from the group consisting of

| | |
|---|---|
| AAAYAAAAAAKAAAA; | (SEQ ID NO:1) |
| AEKYAAAAAAKAAAA; | (SEQ ID NO:2) |
| AKEYAAAAAAKAAAA; | (SEQ ID NO:3) |
| AKKYAAAAAAKAAAA; | (SEQ ID NO:4) |
| AEAYAAAAAAKAAAA; | (SEQ ID NO:5) |
| KEAYAAAAAAKAAAA; | (SEQ ID NO:6) |
| AEEYAAAAAAKAAAA; | (SEQ ID NO:7) |
| AAEYAAAAAAKAAAA; | (SEQ ID NO:8) |
| EKAYAAAAAAKAAAA; | (SEQ ID NO:9) |
| AAKYEAAAAAKAAAA; | (SEQ ID NO:10) |
| AAKYAEAAAAKAAAA; | (SEQ ID NO:11) |
| EAAYAAAAAAKAAAA; | (SEQ ID NO:12) |
| EKKYAAAAAAKAAAA; | (SEQ ID NO:13) |
| EAKYAAAAAAKAAAA; | (SEQ ID NO:14) |
| AEKYAAAAAAAAAAA; | (SEQ ID NO:15) |
| AKEYAAAAAAAAAAA; | (SEQ ID NO:16) |
| AKKYEAAAAAAAAAA; | (SEQ ID NO:17) |
| AKKYAEAAAAAAAAA; | (SEQ ID NO:18) |
| AEAYKAAAAAAAAAA; | (SEQ ID NO:19) |
| KEAYAAAAAAAAAAA; | (SEQ ID NO:20) |
| AEEYKAAAAAAAAAA; | (SEQ ID NO:21) |
| AAEYKAAAAAAAAAA; | (SEQ ID NO:22) |
| EKAYAAAAAAAAAAA; | (SEQ ID NO:23) |
| AAKYEAAAAAAAAAA; | (SEQ ID NO:24) |
| AAKYAEAAAAAAAAA; | (SEQ ID NO:25) |
| EKKYAAAAAAAAAAA; | (SEQ ID NO:26) |
| EAKYAAAAAAAAAAA; | (SEQ ID NO:27) |
| AEYAKAAAAAAAAAA; | (SEQ ID NO:28) |
| AEKAYAAAAAAAAAA; | (SEQ ID NO:29) |
| EKYAAAAAAAAAAAA; | (SEQ ID NO:30) |
| AYKAEAAAAAAAAAA; | (SEQ ID NO:31) |
| AKYAEAAAAAAAAAA. | (SEQ ID NO:32) |

According to one embodiment, the method of the invention further comprises administering the at least one copolymer in combination with at least one immunomodulating agent, the at least one immunomodulating agent being capable of attenuating the activity or presence of Th1 lymphocytes. According to another embodiment, the at least one immunomodulating agent is an antibody selected from the group consisting of α-IL-4, α-IL-12, α-IL-18, α-IFN-γ, α-integrin and α-CD4⁺. According to yet another embodiment, the immunomodulating agent is capable of reducing the expression of molecules selected from the group consisting of: IL-12, IL-18, integrin and IFN-γ. According to yet another embodiment, the immunomodulating agent is selected from the group consisting of: antisense nucleotide sequence, sense nucleotide sequence, ribozyme, interfering RNA and aptamer.

The combination of drugs may be administered together or may be administered sequentially. It is to be explicitly understood that present invention encompasses co-administration of these agents in a substantially simultaneous manner, as in a single unit dosage form suitable for oral or parenteral administration having a fixed ratio of these active agents or in multiple, separate unit dosage forms for each agent, each of which may independently be in a form suitable for oral administration or parenteral injection. It is further to be explicitly understood that present invention explicitly encompasses separate administration including separate schedules or even alternating schedules.

According to various embodiments of the present invention, the therapeutically effective amount of the at least one copolymer ranges from about 1.0 mg to about 500.0 mg/day. Alternatively, such therapeutically effective amounts of the at least one copolymer are from about 20.0 mg to about 100.0 mg/day.

According to one embodiment, the cardiovascular disease and disorder is selected from the group consisting of atherosclerosis and myocarditis.

Although the present specification describes some preferred embodiments of the invention, it is to be understood that the present invention encompasses the use of any synthetic random or ordered copolymer of at least three of Glu or Asp, Lys or Arg, Ala Gly or Val, and Phe or Tyr or Trp, having a relative molar ratio of the amino acid residues and an average molecular weight as defined herein, including those forms of Cop 1 described in the literature that fall within the definition of the present invention.

In another aspect, the invention relates to the use of the at least one copolymer described above in for the manufacture of a medicament for the prevention and treatment of cardiovascular diseases and disorders.

These and further embodiments will be apparent from the detailed description and examples that follow.

### BRIEF DESCRIPTION OF THE DRAWINGS

FIG. 1 demonstrates the average body weights of C57BL/J6 mice, during 10 weeks treatment with PBS (groups A and C; circles and triangles, respectively) or with glatiramer acetate (group B; squares).

FIG. 2 shows lipid concentrations in plasma samples derived from groups A-C (black, gray and white, respectively) of the C57BL/J6 mice.

FIGS. 3A-3C represent microsections of aortic sinus, derived from C57BL/J6 mice (groups A-C) and stained with oil red O.

FIGS. 4A and 4B exhibit the average area (4A) and the relative area, with respect to group B (4B), of aortic sections from mice of group A stained with oil red O.

FIG. 5 demonstrates the average body weights of apoE-/- mice during 8 weeks treatment with PBS or with glatiramer acetate.

FIG. 6 shows cholesterol (black) and triglycerides (TG; white) concentrations in plasma samples derived from apoE-/- mice daily administered with glatiramer acetate or with PBS.

FIG. 7 shows the progression of atherosclerotic lesion area in the aortic sinus of apoE-/- mice treated with PBS (black), low-dose of glatiramer acetate (gray), or high-dose of glatiramer acetate (dark gray).

FIG. 8 shows the percentage of lesions of aortic area in apoE-/- mice treated with PBS or with glatiramer acetate.

### DETAILED DESCRIPTION OF THE INVENTION

The present invention relates to the use of random or ordered copolymers including Copolymer 1 and Copolymer 1-related heteropolymers or ordered peptides, for treating or preventing cardiovascular diseases and disorders.

Definitions

The term “treatment and prevention of cardiovascular disease” as used herein refers to cardiac therapy as well as the prevention and/or treatment of other diseases associated with the cardiovascular system, such as heart disease. The term “heart disease” as used herein refers to any type of disease, disorder, trauma or surgical treatment that involves the heart or myocardial tissue. Of particular interest are heart diseases that relate to hypoxia and/or ischemia of myocardial tissue and/or heart failure. One type of heart disease that can result from ischemia is reperfusion injury, such as can occur when anti-coagulants, thrombolytic agents, or anti-anginal medications are used in therapy, or when the cardiac vasculature is surgically opened by angioplasty or by coronary artery grafting. Another type of heart disease to which the invention is directed is coronary artery disease (CAD), which can arise from arteriosclerosis, particularly atherosclerosis, a common cause of ischemia. CAD has symptoms such as stable or unstable angina pectoris, and can lead to acute myocardial infarctions (AMI) and sudden cardiac death.

Although the term “atherosclerosis” is one type of arteriosclerosis, this term as used herein refers to any disease that is generically defined as atherosclerosis (or “hardening of the arteries”). The terms “atherosclerosis” and the generic term “arteriosclerosis” are interchangeably used herein to describe a number of cardiovascular diseases including diseases in which the arterial wall becomes thickened and loses elasticity and myocarditis which is often associated and moreover induced by the coxsackievirus B. Atherosclerosis involves a process that causes a build-up of deposits on artery walls called plaque. Typically, the deposits occur in the tunica intima (the innermost layer of a blood vessel) of large and medium-sized arteries. The plaques contain fatty substances, cholesterol, cellular waste products, fibrin found in blood), and calcium. Plaque can become large enough to partially or totally block the flow of blood through an artery. A build-up of plaque or a rupture occurring within the plaque can result in a blood clot. The dislodged plaque material can travel to other parts of the body (e.g., brain, heart, kidneys, and legs), resulting in serious injury to tissues and organs, principally by blocking blood flow through smaller arteries.

The term “therapeutically effective amounts” is intended to qualify the amount of copolymer that will achieve the goal of treating or preventing cardiovascular diseases and disorders.

### PREFERRED MODES FOR CARRYING OUT THE INVENTION

Atherosclerosis is a progressive, complex disease often associated with the aging process, though early signs of atherosclerosis have been identified also in children. Atherosclerosis is a factor in several conditions including coronary heart disease (CHD), myocardial infarction (Ml), angina pectoris, cerebral vascular disease (CVD), thrombotic stroke, transient ischemic attacks (TIAs), insufficient blood supply to lower limbs and feet (claudication), organ damage, and vascular complications of diabetes. The symptoms of atherosclerosis can be few or minor in the early stages. It can progress undetected for years, particularly in individuals who are at high risk for heart disease and accordingly atherosclerosis is referred to as “the silent killer”. Moreover, about half of the children and siblings of individuals with diseased coronary arteries show signs of atherosclerosis, indicating that hereditary factors play an important role in the onset of this disease. Thus, there is an unmet need to prevent atherosclerosis in subjects with high risk of having this disease, such as family members of a subject with diseased coronary arteries. Furthermore, as most of the drugs commonly used for treating atherosclerosis are merely directed to alleviate the symptoms associated with this disease, there is an unmet need for therapeutic modalities directed to treat atherosclerosis at its early stages.

The present invention overcomes the drawbacks of the background art by providing a novel method for preventing arteriosclerosis or treating this disease at its early stages by administering therapeutically effective amount of a copolymer selected from Cop 1 or Cop 1-related heteropolymers or ordered peptides, alone or in combination with immunomodulating agents, to a subject in need thereof wherein the copolymer correspond to one of the following copolymers:

A. A copolymer comprising at least three amino acids selected from at least three of the following groups:
(a) lysine and arginine;
(b) glutamic acid and aspartic acid;
(c) alanine, glycine and valine;
(d) tyrosine, tryptophan and phenylalanine;

B. A copolymer comprising at least three different amino acids, each selected from a different group selected from:
(a) lysine and arginine;
(b) glutamic acid and aspartic acid;
(c) alanine, glycine and valine;
(d) phenylalanine, tyrosine and tryptophan;

C. A copolymer comprising alanine, glutamic acid, lysine, and tyrosine, of net overall positive electrical charge:

D. Copolymer 1 comprising alanine, glutamic acid, lysine, and tyrosine in the molar ratios of glutamic acid about 0.14 to alanine about 0.43 to tyrosine about 0.10 to lysine about 0.34;

E. Copolymer 1 comprising alanine, glutamic acid, lysine, and tyrosine in the molar ratios of: 0.17 glutamic acid to 0.38 lysine to 0.49 alanine to 0.10 tyrosine;

F. Copolymer 1 comprising alanine, glutamic acid, lysine, and tyrosine in the molar ratios of: 0.19 glutamic acid to 0.4 lysine to 0.6 alanine to 0.1 tyrosine;

G. Anyone of the aforementioned copolymer having an average molecular weight of about 2,000-40,000 Da; preferably, about 13,000-18,000 Da.

H. Glatiramer acetate having an average molecular weight of about 5,000-9,000 Da;

I. Terpolymer having three different amino acids each one selected from groups (a) to (d);

J. Ordered terpolymer consisting of three different amino acids, each selected from a different one of the following groups:
(a) lysine and arginine;
(b) glutamic acid and aspartic acid;
(c) phenylalanine, tyrosine and tryptophan;

K. A terpolymer YEK substantially free of alanine and containing glutamic acid, tyrosine, and lysine, in the molar ratio of from about 0.005 to about 0.300 glutamic acid, from about 0.005 to about 0.250 tyrosine, and from about 0.3 to about 0.7 lysine;

L. A terpolymer YEK substantially free of alanine and containing glutamic acid, tyrosine, and lysine, in the molar ratio of about 0.26 to about 0.16 to about 0.58, respectively.

M. YEK as detailed above, with an average molecular weight of about 2,000-40,000 Da, preferably about 13,000-18,000 Da, wherein it is possible to substitute arginine for lysine, aspartic acid for glutamic acid or phenylalanine or tryptophan for tyrosine.

N. A YAK terpolymer consisting of three different amino acids, each selected from a different one of the following groups:
(a) lysine and arginine;
(b) alanine, glycine and valine;
(c) phenylalanine, tyrosine or tryptophan;

O. A YAK terpolymer substantially free of glutamic acid containing tyrosine, alanine and lysine, in the molar ratio of from about 0.005 to about 0.25 tyrosine, from about 0.3 to about 0.6 alanine, and from about 0.1 to about 0.5 lysine, alternatively, the molar ratios of tyrosine, alanine and lysine are about 0.10 to about 0.54 to about 0.35, respectively;

P. YAK as detailed above, with an average molecular weight of about 2,000-40,000 Da, preferably about 13,000-18,000 Da wherein it is possible to substitute arginine for lysine, glycine or valine for alanine or phenylalanine or tryptophan for tyrosine;

Q. A YEA terpolymer consisting of three different amino acids, each selected from a different member of the following groups:
(a) glutamic acid and aspartic acid;
(b) alanine, glycine and valine;
(c) phenylalanine, tyrosine and tryptophan;

R. YEA terpolymer containing tyrosine, glutamic acid and alanine, in the molar ratio of from about 0.005 to about 0.25 tyrosine, from about 0.005 to about 0.3 glutamic acid, and from about 0.005 to about 0.8 alanine, alternatively, the molar ratios of glutamic acid, alanine, and tyrosine are about 0.21 to about 0.65 to about 0.14, respectively; and

S. YEA as detailed above, having average molecular weight of about 2,000-40,000 Da, preferably about 13,000-18,000 Da, wherein it is possible to substitute aspartic acid for glutamic acid, glycine for alanine, and phenylalanine or tryptophan for tyrosine.

Anyone of the foregoing copolymers used in the method of the present invention may have random amino acid sequence (random copolymers), ordered amino acid sequence (ordered copolymers) or peptides having ordered amino acid sequence (ordered peptides).

Moreover, the copolymers for use in the present invention may be composed of L- or D-amino acids or mixtures thereof. As is known by those of skill in the art, L-amino acids occur in most natural proteins. However, D-amino acids are commercially available and can be substituted for some or all of the amino acids used to make the terpolymers and other copolymers of the present invention. The present invention contemplates copolymers containing both D- and L-amino acids, as well as copolymers consisting essentially of either L- or D-amino acids.

In a preferred embodiment, the mole fraction of amino acids of the heteropolymers for use in the method of the invention is about what is preferred for Copolymer 1. The mole fraction of amino acids in Copolymer 1 is glutamic acid about 0.14, alanine about 0.43, tyrosine about 0.10, and lysine about 0.34. According to certain embodiments, the average molecular weight for Copolymer 1 is between about 13.000 and about 18,000 daltons. The activity of Copolymer 1 in the treatment or prevention of atherosclerosis is expected to remain if one or more of the following substitutions are made: aspartic acid for glutamic acid, glycine for alanine, and arginine for lysine

Copolymer 1 has been approved in several countries for the treatment of Multiple Sclerosis (MS) under the trade name, Copaxone®, Glatiramer acetate. Several clinical trials demonstrated that Copolymer 1 is well tolerated with only minor side reactions which were mostly mild reactions at the injection site (Johnson et al., Neurology, 1:65,1995).

The molar ratios of the monomers of the more preferred terpolymer of glutamic acid, alanine, and tyrosine, or YEA, is about 0.21 to about 0.65 to about 0.14.

The molar ratios of the monomers of the more preferred terpolymer of glutamic acid, tyrosine, and lysine, or YEK, is about 0.26 to about 0.16 to about 0.58.

The molar ratios of the monomers of the more preferred terpolymer of tyrosine, alanine and lysine, or YAK, is about 0.10 to about 0.54 to about 0.35.

As indicated hereinabove, heteropolymers having ordered amino acid sequence (ordered copolymers) are within the scope of the present invention. Examples of such heteropolymers and peptides are those disclosed in WO 00/05249, the entire contents of which being hereby incorporated herein by reference. Thirty-two of the peptides specifically disclosed in said application are reproduced in Table 1, hereinbelow. Such peptides and other similar peptides are expected to have similar activity as Cop 1. Such peptides, and other similar peptides, are also considered to be within the definition of Cop 1-related peptides or polypeptides and their use is considered to be part of the present invention.

TABLE 1
ORDERED PEPTIDES

| | |
|---|---|
| SEQ ID NO. | Peptide Sequence |

| | |
|---|---|
| 1 | AAAYAAAAAAKAAAA |
| 2 | AEKYAAAAAAKAAAA |
| 3 | AKEYAAAAAAKAAAA |
| 4 | AKKYAAAAAAKAAAA |
| 5 | AEAYAAAAAAKAAAA |
| 6 | KEAYAAAAAAKAAAA |
| 7 | AEEYAAAAAAKAAAA |
| 8 | AAEYAAAAAAKAAAA |
| 9 | EKAYAAAAAAKAAAA |
| 10 | AAKYEAAAAAKAAAA |
| 11 | AAKYAEAAAAKAAAA |
| 12 | EAAYAAAAAAKAAAA |
| 13 | EKKYAAAAAAKAAAA |
| 14 | EAKYAAAAAAKAAAA |
| 15 | AEKYAAAAAAAAAAA |
| 16 | AKEYAAAAAAAAAAA |
| 17 | AKKYEAAAAAAAAAA |
| 18 | AKKYAEAAAAAAAAA |
| 19 | AEAYKAAAAAAAAAA |
| 20 | KEAYAAAAAAAAAAA |
| 21 | AEEYKAAAAAAAAAA |
| 22 | AAEYKAAAAAAAAAA |
| 23 | EKAYAAAAAAAAAAA |
| 24 | AAKYEAAAAAAAAAA |
| 25 | AAKYAEAAAAAAAAA |
| 26 | EKKYAAAAAAAAAAA |
| 27 | EAKYAAAAAAAAAAA |
| 28 | AEYAKAAAAAAAAAA |
| 29 | AEKAYAAAAAAAAAA |
| 30 | EKYAAAAAAAAAAAA |
| 31 | AYKAEAAAAAAAAAA |
| 32 | AKYAEAAAAAAAAAA |

As disclosed herein for the first time and exemplified herein below, exogenous subcutaneous administration of Glatiramer Acetate inhibits the development of atherosclerosis in C57BL/6J mice maintained on an atherogenic diet.

Furthermore, subcutaneous and oral administrations of Glatiramer Acetate inhibit the progression of atherosclerosis in apolipoprotein E-deficient (apoE-/-) mice and reduce atherosclerotic lesions.

ApoE-/- mice are well-established atherosclerotic models. Similar to the atherosclerotic lesion of humans, T lymphocytes are present in apoE-/- lesion (Roselaar et al., 1996, Artherioscler Thromb Vasc Biol 16:1013-1018) and immunomodulation by either oral tolerance or immunization have been shown to influence the disease (George et al., 2004, Cardiovasc Res. 62:603-9). The present invention discloses for the first time the anti-atherogenic effect of Glatiramer Acetate, which is currently used for treatment of multiple sclerosis in clinical practice.

In order to verify the anti-atherogenic effect of Glatiramer Acetate during the early development of atherosclerosis, the hyperlipidemic model of the disease in C57BL/6J male mice (Varadhachary, ibid) and the apoE-/- mice atherosclerotic model were used. Accumulating evidence indicates that Th1 immune response associated with atherosclerosis is deleterious and that a modulation of the Th1 differentiation pathway may provide a new pharmacological tool to treat this disease. Thus, it was recently shown that a 12 week treatment with pentoxifylline (PTX), a known phosphodiesterase inhibitor of the Th1 differentiation pathway, decreased the size of atherosclerotic lesions by 60% in apolipoprotein E-deficient mice (Laurat et al., 2001, 104:197-202). As shown in the Examples, daily administration of GA for 8 weeks, a period sufficient for GA-induced Th1 to Th2 shift, led to a marked decreased in the area of fatty streaks as compared to the non-treated group. Most previous attempts to investigate different aspects of immunomodulation on atherosclerosis were carried out in genetically immunocompromised animals such as mice deficient of apolipoprotein E, LDL receptor, IL-10, or INF-γ fed with normal or atherogenic chow (e.g. Whitman 2000, ibid). However, some investigations of the effect of different exogenously administered compounds on the development of atherosclerosis were done in the model of susceptible C57BL/6J mouse strain maintained on atherogenic diet (Garber et al., 2001, J. Lipid Res. 42, 545-552; Gonen et al., 2002-03, 70: 215-218), focusing on reducing atherosclerosis by non-immunomodulatory treatment, and showing a decrease in the development of atherosclerosis in spite of a lack of effect on blood lipid levels, which were elevated in both treated and non-treated groups.

The present invention demonstrates a combined concept of using immunomodulation with an existing drug (indicated for multiple sclerosis) to inhibit atherosclerosis in susceptible mice maintained on atherogenic diet. The anti-atherosclerotic effect was achieved despite the fact that the plasma cholesterol and LDL levels, that were significantly elevated in the groups that received atherogenic chow in comparison to the control group, were not affected by the GA treatment. This result, as well as the finding that the body weight of the mice was not affected by the atherogenic diet is consistent with the previous findings (Garber et al. and Gonen et al., ibid).

The present invention further discloses that Glatiramer Acetate has a potential to prevent atherosclerosis.

Without wishing to be bound by any theory or mechanism, the effect of GA in decreasing the aortic fatty streak area may be attributed to its capacity to inhibit the proliferation and secretion of Th1 cytokines while enhancing the secretion of Th2 cytokines, as observed in mice with experimental autoimmune encephalomyelitis (EAE) (Aharoni, 2001, Transplantation, 72:598-605). Moreover, an increase in IL-10 and IL-4 levels and a decrease in IL-12 and tumor necrosis factor-alpha (TNF-α) levels are normally seen with GA administration in patients suffering of MS (Salama et al., 2003, Brain, 126:2638-2647). Such increased IL-10 secretion originated by Th2 cells, B cells, and macrophages indeed led to reductions in atherosclerotic lesion size (Varadhachary, ibid). Furthermore, the overexpression of IL-10 attenuated the formation of atherosclerotic lesions and was associated with alterations in lymphocyte and inacrophage phenotypes (Pinderski et al., 2002, Circ Res. 90, 1064-1071). It may, therefore be presumed that in atherogenesis as well there is a potential therapeutic benefit by increasing IL-10 expression, either locally or systemically, a well established effect of GA. TGF-β, another Th2 cytokine elevated by GA (Aharoni, ibid), may also play a role in affecting atherosclerosis. It was suggested that one of the immune activities of TGF-β is to modulate the CD4⁺ effector pathway, as evidenced by the attenuation of Th1-type responses in vivo, which led to the consequent suggestion that the immunomodulating effects of TGF-β may regulate inflammatory responses in atherosclerotic conditions (Koglin et al., 1998, Circ Res. 83:652-660).

### Combined Therapies

According to various embodiments, the method of the invention further comprises administering the at least one copolymer in combination with at least one immuinomodulating agent, the at least one immunomodulating agent is capable of attenuating the activity or presence of Th1 lymphocytes. According to another embodiment, the at least one immunomodulating agent is an antibody selected from the group consisting of: α-IL-4, α-IL-12, α-IL-18, α-IFN-γ, α-integrin and α-CD4⁺. The α-integrin may be any molecule that is capable of preventing infiltration of lymphocytes.

According to yet another embodiment, the immunomodulating agent is capable of inhibiting the expression of molecules selected from the group consisting of IL-12, IL-18, integrin and IFN-γ. The immunomodulating agent may thus be a polynucleotide selected from the group consisting of antisense nucleotide sequence, sense nucleotide sequence, short interfering RNA, ribozyme and aptamer.

In recent years, advances in nucleic acid chemistry and gene transfer have inspired new approaches for treating diseases by gene interference. Antisense technology has been one of the most commonly described approaches in protocols to achieve gene-specific interference. For antisense strategies, stoichiometric amounts of single-stranded nucleic acid complementary to the messenger RNA for the gene of interest are introduced into the cell.

International Publication No. WO 02/10365 provides a method for gene suppression in eukaryotes by transformation with a recombinant construct containing a promoter, at least one antisense and/or sense nucleotide sequence for the gene(s) to be suppressed. Antisense oligonucleotide therapies for certain cancerous conditions are described in U.S. Pat. No. 5,098,890. U.S. Pat. No. 5,135,917 provides antisense oligonucleotides that inhibit human interleukin-l receptor expression. U.S. Pat. No. 5,087,617 provides methods for treating cancer patients with antisense oligonucleotides. U.S. Pat. No. 5,166,195 provides oligonucleotide inhibitors of HIV. U.S. Pat. No. 5,004,810 provides oligomers capable of hybridizing to herpes simplex virus Vmw65 mRNA and inhibiting replication. U.S. Pat. No. 5,194,428 provides antisense oligonucleotides having antiviral activity against influenza virus. U.S. Pat. No. 4,806,463 provides antisense oligonucleotides and methods using them to inhibit HTLV-III (human T-cell lymphotropic virus type III, known also as HIV) replication. Antisense oligonucleotides have been safely administered to humans and several clinical trials of antisense oligonucleotides are presently underway. It is, thus, established that oligonucleotides can be useful therapeutic instrumentalities and that the same can be configured to be useful in treatment regimes for treatment of cells and animals, especially humans.

Aptamers are specifically binding oligonucleotides for non-oligonucleotide targets that generally bind nucleic acids. The use of single-stranded DNA as an appropriate material for generating aptamers is disclosed in U.S. Pat. No. 5,840,567. Use of DNA aptamers has several advantages over RNA including increased nuclease stability, in particular plasma nuclease stability, and ease of amplification by PCR or other methods. RNA generally is converted to DNA prior to amplification using reverse transcriptase, a process that is not equally efficient with all sequences, resulting in loss of some aptamers from a selected pool.

RNA interference, using short interfering RNAs (siRNAs; Fire et al., Nature 391:806, 1998), refers to the process of sequence-specific post-transcriptional gene silencing in animals mediated by siRNAs. The corresponding process in plants is commonly referred to as post-transcriptional gene silencing or RNA silencing. The process of post-transcriptional gene silencing is thought to be an evolutionarily conserved cellular defense mechanism used to prevent the expression of foreign genes and is commonly shared by diverse flora and phyla.

RNA interference involves application of double stranded RNA (dsRNA) that reduces the expression of the gene to which the dsRNA corresponds. The phenomenon of RNAi was subsequently proven to exist in many organisms and to be a naturally occurring cellular process. International Publication No. WO 00/01846 discloses methods for identifying specific genes responsible for conferring a particular phenotype in a cell using specific dsRNA molecules. International Publication No. WO 01/29058 discloses specific genes involved in dsRNA-mediated RNAi. International Publication No. WO 99/07409 discloses specific compositions consisting of particular dsRNA molecules combined with certain anti-viral agents. International Publication No. WO 99/53050 discloses certain methods for decreasing the phenotypic expression of a nucleic acid in plant cells using certain dsRNAs. International Publication No. WO 01/49844 discloses specific DNA constructs for use in facilitating gene silencing in targeted organisms.

International Publications Nos. WO 02/055692, WO02/055693, and EP 1144623 B1 disclose methods for inhibiting gene expression using RNAi. International Publications Nos. WO 99/49029 and WO01/70949, and AU 4037501 describe certain vectors expressing siRNA molecules. U.S. Pat. No. 6,506,559, discloses methods for inhibiting gene expression in vitro using certain siRNA constructs that mediate RNAi. U.S. Pat. No. 5,681,747 discloses methods for inhibiting human-PKCα expression with an oligonucleotide specifically hybridizable to a portion of the 3′-untranslated region of PKCα.

The combination of drugs may be administered together or may be administered sequentially. It is to be explicitly understood that present invention explicitly encompasses co-administration of these agents in a substantially simultaneous manner, as in a single unit dosage form suitable for oral or parenteral administration having a fixed ratio of these active agents or in multiple, separate unit dosage forms for each agent, each of which may independently be in a form suitable for oral administration or parenteral injection.

The copolymers can be made by any procedure available to one of skill in the art. For example, the copolymers can be made under condensation conditions using the desired molar ratio of amino acids in solution, or by solid phase synthetic procedures. Condensation conditions include the proper temperature, pH, and solvent conditions for condensing the carboxyl group of one amino acid with the amino group of another amino acid to form a peptide bond. Condensing agents, for example, dicyclohexyl-carbodiimide, can be used to facilitate the formation of the peptide bond. Blocking groups can be used to protect functional groups, such as the side chain moieties and some of the amino or carboxyl groups against undesired side reactions.

The process disclosed in U.S. Pat. No. 3,849,550, can be used for preparing the copolymers of the invention. For example, the N-carboxyanhydrides of tyrosine, alanine, γ-benzyl glutamate and N, ε-trifluoroacetyl-lysine are polymerized at ambient temperatures in anhydrous dioxane with diethylamine as an initiator. The γ-carboxyl group of the glutamic acid can be deblocked by hydrogen bromide in glacial acetic acid. The trifluoroacetyl groups are removed from lysine by one molar piperidine. One of skill in the art readily understands that the process can be adjusted to make peptides and polypeptides containing the desired amino acids, that is, three of the four amino acids in Copolymer 1, by selectively eliminating the reactions that relate to any one of glutamic acid, alanine, tyrosine, or lysine. U.S. Pat. Nos. 6,620,847; 6,362,161; 6,342,476; 6,054,430; 6,048,898 and 5,981,589 disclose improved methods for preparing glatiramer acetate (Cop-1). For purposes of this application, the terms “ambient temperature” and “room temperature” typically means a temperature ranging from about 20° C. to about 26° C.

The molecular weight of the copolymers can be adjusted during polypeptide synthesis or after the terpolymers have been made. To adjust the molecular weight during polypeptide synthesis, the synthetic conditions or the amounts of amino acids are adjusted so that synthesis stops when the polypeptide reaches the approximate length which is desired. After synthesis, polypeptides with the desired molecular weight can be obtained by any available size selection procedure, such as chromatography of the polypeptides on a molecular weight sizing column or gel, and collection of the molecular weight ranges desired. The present polypeptides can also be partially hydrolyzed to remove high molecular weight species, for example, by acid or enzymatic hydrolysis, and then purified to remove the acid or enzymes.

In one embodiment, the copolymers with a desired molecular weight may be prepared by a process, which includes reacting a protected polypeptide with hydrobromic acid to form a trifluoroacetyl-polypeptide having the desired molecular weight profile. The reaction is performed for a time and at a temperature which is predetermined by one or more test reactions. During the test reaction, the time and temperature are varied and the molecular weight range of a given batch of test polypeptides is determined. The test conditions which provide the optimal molecular weight range for that batch of polypeptides are used for the batch. Thus, a trifluoroacetyl-polypeptide having the desired molecular weight profile can be produced by a process which includes reacting the protected polypeptide with hydrobromic acid for a time and at a temperature predetermined by test reaction. The trifluoroacetyl-polypeptide with the desired molecular weight profile is then further treated with an aqueous piperidine solution to form a low toxicity polypeptide having the desired molecular weight.

In a preferred embodiment, a test sample of protected polypeptide from a given batch is reacted with hydrobromic acid for about 10-50 hours at a temperature of about 20-28° C. The best conditions for that batch are determined by running several test reactions. For example. in one embodiment, the protected polypeptide is reacted with hydrobromic acid for about 17 hours at a temperature of about 26° C.

### Pharmaceutical Compositions

The random and ordered copolymers used in the present invention can be formulated into pharmaceutical compositions containing a pharmaceutically acceptable carrier. As used herein, “pharmaceutically acceptable carrier” includes any and all solvents, dispersion media, coatings, antibacterial and antifungal agents, isotonic and absorption delaying agents, sweeteners and the like. The pharmaceutically acceptable carriers may be prepared from a wide range of materials including, but not limited to diluents, binders and adhesives. lubricants, disintegrants, coloring agents, bulking agents, flavoring agents, sweetening agents and miscellaneous materials such as buffers and absorbents that may be needed in order to prepare a particular therapeutic composition. The use of such media and agents with pharmaceutically active substances well known in the art. Except insofar as any conventional media or agent is incompatible with the active ingredient, its use in the therapeutic compositions is contemplated.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example DMSO, or polyethylene glycol are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

Pharmaceutical compositions for parenteral administration include aqueous solutions of the active ingredients in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

U.S. Pat. No. 6,214,791 discloses methods for treating multiple sclerosis by oral administration of copolymer-1 through ingestion or inhalation. When copolymer-1 is introduced orally, it may be mixed with other food forms and consumed in solid, semi-solid, suspension, or emulsion form; and it may be mixed with pharmaceutically acceptable carriers, including water. suspending agents, emulsifying agents, flavor enhancers, and the like. In one embodiment, the oral composition is enterically-coated. Copolymer-1 may also be administered nasally in certain of the above-mentioned forms by inhalation or nose drops. Furthermore, oral inhalation may be employed to deliver copolymer-1 to the mucosal linings of the trachea and bronchial passages.

According to various embodiments of the present invention, the therapeutically effective amount of the at least one copolymer ranges from about 1.0 mg to about 500.0 mg/day. Alternatively, such therapeutically effective amounts of the at least one copolymer are from about 20.0 mg to about 100.0 mg/day.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

Those skilled in the art will appreciate that the foregoing embodiments utilize generally cylindrical configurations, but that other shapes are within the scope of the present invention. Thus, the inlet and/or the first and/or second sections could have polygonal cross-section, in which case the limitations discussed above with respect to the dimensions of the outside diameters would instead apply to the outside perimeters.

### EXAMPLES

### Materials and Methods

### C57BL/J6 Mice

Fourteen-month-old male C57BL/J6 mice (Harlan) with an average body weight of 19 g were randomly divided into three groups (five animals in each group). The groups entered a two-week acclimatization period. Groups A, which served as positive control and B, which served as the treated group, received a high fat atherogenic diet containing 1.25 % cholesterol, 0.5% sodium cholate and 15% fat (Harlan Laboratories, Teklad Premier Laboratory Diets, Madison, Wis., USA TD 88051). Group C served as negative control and was maintained on a normal mouse chow (Altromin, Standard Diet). The chow was supplied considering that the average intake of food for each animal is about 5 g/day (Walker et al., 1999, Arterioscler Thromb Vasc Biol. 19, 2673-2679). Replenishment of food and autoclaved water was performed every other day. All the animals were weighed weekly during the study. The mice were maintained on a 12-hour light/dark cycle in ventilated cages and temperature-controlled environment, in accordance with the standard animal care requirements of the Weizmann Institute of Science.

Following acclimatization, the animals in groups A (positive control) and C (negative control) received 0.2 ml of PBS injected daily subcutaneously (SC). The animals in group B (treated) received SC daily injections of glatiramer acetate (GA) (COPAXONE®, Teva Pharmaceutical Industries Ltd) in PBS (0.5 mg/0.2 ml). After 8 weeks all the mice were bled retro-orbitally and humanely sacrificed by cervical dislocation. The hearts and ascending aorta were removed from the animals, rinsed with Dulbecco's PBS (Life Technologies) and used for the evaluation of fatty streak formation.

### ApoE-/- Mice

apoE-/- (backcrossed 10 times to C57BL/6J) mice were obtained from the Jackson Laboratory and were maintained under specific pathogen-free conditions and fed a normal chow diet. All experimental procedures were performed with approval from the Animal Care Committee of the Weizmann Institute of Science.

Eight-month-old apoE-/- mice (101 mice) were divided into 7 groups, 15 in each group (except day 0 group; n=11). Control group-time 0 (group 1) was sacrificed before treatment in order to measure the atherosclerotic lesion area prior to the treatments. Treatments were performed daily for a period of 8 weeks. Control group (group II) was orally administrated with PBS (0.2 ml). Control group III was injected subcutaneously (SC) with PBS (0.2 ml). Group IV was orally treated with GA in PBS (5.0 μg/0.2 ml); Groups V was orally treated with GA in PBS (250 μg/0.2 ml). Group VI was injected subcutaneously (SC) with GA in PBS (0.5 mg/0.2 ml). Group VII was injected subcutaneously (SC) with GA in PBS (2.0 mg/0.2 ml).

### Plasma Lipid Concentration

The whole blood samples were centrifuged for plasma separation and the plasma samples of each group were pooled. Total Plasma cholesterol as well as low and high density lipoprotein cholesterol (LDL and HDL, respectively) concentrations were measured in each group using Konelab 30i autoanalyser (Kone Instruments, Espoo, Finland).

### Quantitation of Atherosclerotic Fatty Streak Area

Histological evaluations of aortic fatty streak formation were performed according to the method of Paigen et al. with some modifications (Paigen et al., 1987. Atherosclerosis. 68:231-240). Briefly, the apical two thirds of the myocardium were removed and the basal part was snap-frozen in OCT (Sakura Tissue-Tek) using Isopentan cooled in liquid nitrogen. The frozen tissue blocks were placed on a cryostat, and 10-μm serial slices of the ascending aorta were collected on charge-coated Superfrost glass slides until it became possible to locate the most cranial portion of the aortic sinus by examining unstained slices. Once this slice was identified, 40 slices covering 400 μm of the ascending aorta were collected. Every second slice of the 40 collected was stained with oil red O (Riedel de Haen), counterstained with hematoxylin (Merck) stains and used for further evaluation. The fatty streak area stained with oil red O was objectively determined using a computer microscopy planimetry system (Nikon Micro & Macro system eclipse E800; Nikon digital camera DXM 1200 CCD; Nikon ×20/0.75 lens magnification) and Image-Pro Plus image analyses software for Windows XP. The total stained area (calculated for all 20 slices of each animal) was averaged per section for each animal. Thereafter, the area per slice was averaged for each group of animals and presented in μm².

### Statistical Analyses

ANOVA single factor test was used for the comparison of the body weights of the C57BL/J6 mice. Since no fatty streaks were detected in the negative control group of the C57BL/J6 mice (Group C), only two groups (A and B) participated in the statistical analysis of the results obtained in the quantization of aortic fatty streak area. The C57BL/J6 data were analyzed using Student's t-test: two samples assuming unequal variance. The results are presented as mean ±standard deviation (SD) with a p≦0.05 considered significant.

Student's t-test was used for the comparison of the lesion progression in the aortic sinus of the treated and control groups of the apoE-/- mice.

### Example 1

### Effect of Glatiramer Acetate (GA) Treatment on Survival and Body Weight of CS7BL/J6 Mice

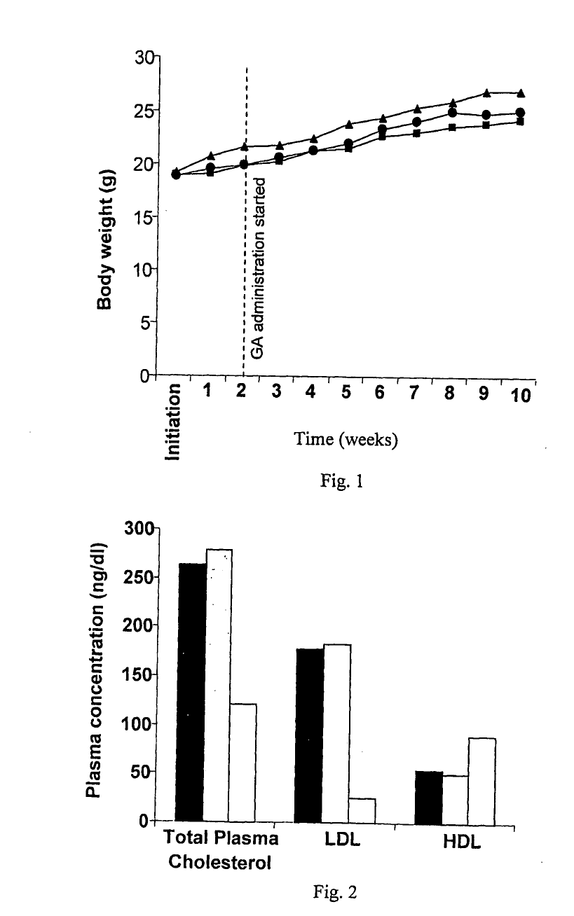

The average body weights recorded for the groups in the beginning of the study were 18.82±1.12, 18.96±1.28, and 19.10±1.14 g for Groups A (positive control), B (treated) and C (negative control), respectively (FIG. 1). Ten weeks following the initiation of the study, the average body weights were 24.98±1.93, 24.20±0.94, and 26.90±2.17 g for Groups A, B and C, respectively (FIG. 1). Thus, the weight gained by the animals during the 10 weeks of the study was 6.16±2.93, 5.24±1.40, and 7.80±2.20 g for Groups A, B and C, respectively. Five additional mice were maintained on the cholesterol rich diet to calibrate the development of lipid streaks. After the initiation of the study, the animals in Groups A, and B, fed with atherogenic chow, gained on the average less weight than the animals in Group C fed with normal mouse chow. However, no statistical difference was found in either the initial body weight, in the body weight following 10 weeks, or the weight gained during the study. Following continuous loss of body weight, one animal in the GA-treated Group B and one mouse from the additional atherogenic diet fed group were found dead five and six weeks into the study, respectively. PM analyses did not reveal any abnormalities in these animals. Four out of five animals in Group B and all five animals in Groups A and C reached the end of the study.

### Example 2

### Effect of Glatiramer Acetate (GA) Treatment on Plasma Lipid Concentration of C57BL/J6 Mice

Total plasma cholesterol concentration measured in Groups A and B was 264 and 279 mg/dl, respectively, and it was more than twice as high as in Group C (FIG. 2). LDL concentration measured in Groups A and B was 178 and 183 mg/dl, respectively, which was more than 7 times higher than in Group C. HDL concentration measured in Groups A and B was 54 and 51 mg/dl, which was lower than as in Group C (FIG. 2). Thus, the atherogenic diet led to increase in cholesterol and LDL, and decrease in HDL concentrations. GA treatment had no effect on the lipid concentrations.

### Example 3

### Effect of Glatiramer Acetate (GA) Treatment on Atherosclerotic Fatty Streak Area of C57BL/J6 Mice

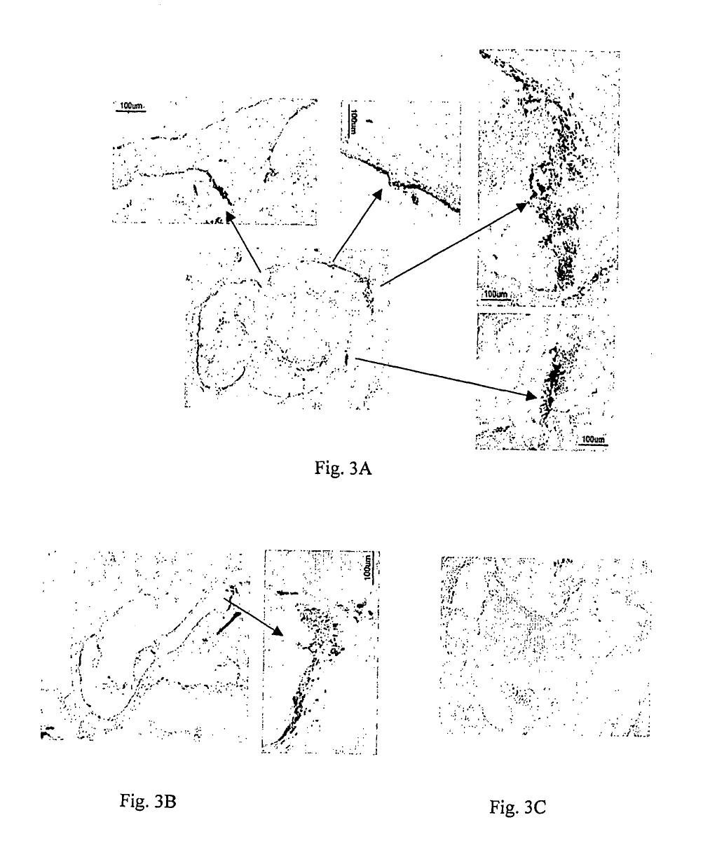

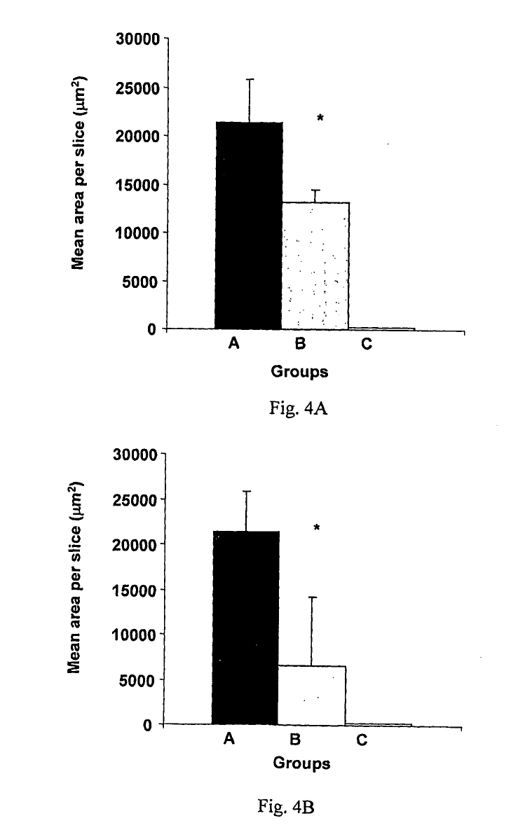

All five mice in Group A, which were fed on the cholesterol rich diet. developed fatty streaks in their aorta (FIG. 3A). In contrast, no fatty streaks were found in the analysed aortic section (20 slices) in any of the five animals in the control Group C (FIG. 3C) and in two out of four animals that reached the end of the study in the GA-treated Group B (FIG. 3B). As shown in FIG. 4A, the average area stained with oil red O and quantified by computer microscopy, in the two GA-treated mice that had fatty streaks, was significantly smaller (only 40%) than that in the positive control Group A (p=0.014). If calculated in comparison to all four mice in Group B, the reduction in the fatty streak area was 4 fold compared to the untreated mice (FIG. 4B). In these figures the symbol (*) indicates a significant difference from the non-treated group (p≦0.05).

### Example 4

### Effect of Glatiramer Acetate (GA) Treatment on the Progression of Atherosclerosis in ApoE-/- Mice

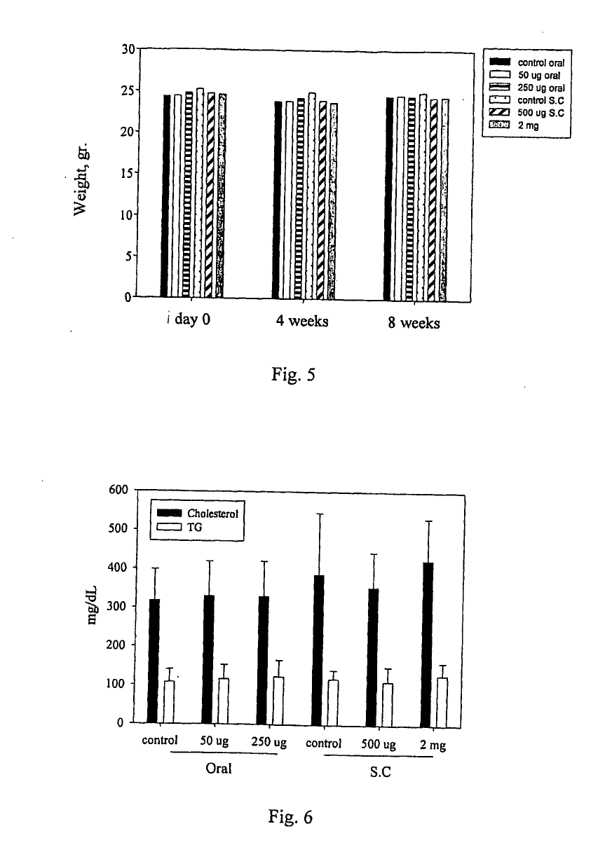

High doses and low doses of GA, administered subcutaneously or orally, did not affect body weight gain, plasma triglycerides (TG) and plasma cholesterol levels in apoE-/- mice (FIGS. 5-6).

Atherosclerotic lesion progression in the aortic sinus was calculated for control and treated groups. A significant increase in lesion area was detected in group III (S.C.

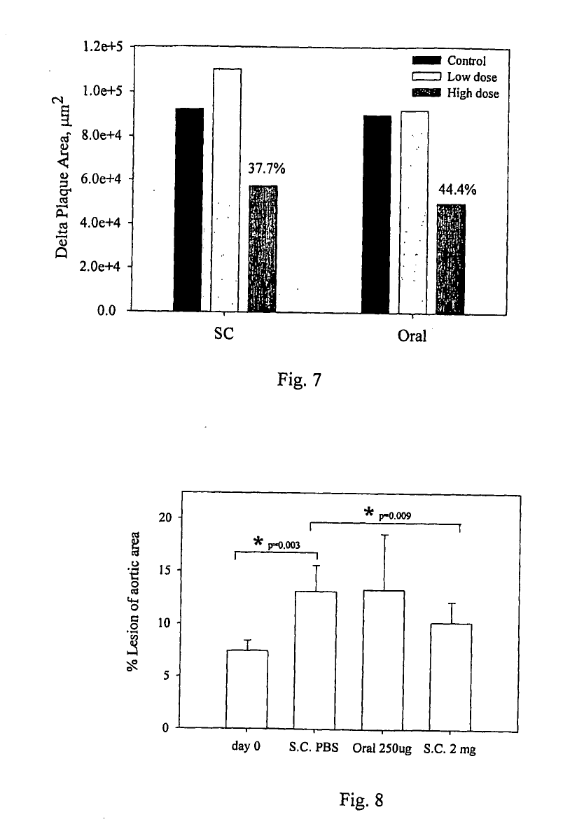

PBS) compared to group I (time 0) (92,425 μm², p=0.046). Low doses of GA administred orally (50 μg) or S.C. (500 μg) did not effect lesion progression, while both high-doses administered orally or S.C. (250 μg and 2 μg, respectively) inhibited the progression of lesion area in substantially the same manner, i.e. by 37.7% and 44.4%, respectively, with respect to control (FIG. 7).

Aortic atherosclerotic areas were measured in control and treated mice of the following groups: I (time 0); III (S.C. PBS); V (Oral GA, 250 μg); and VII (S.C. GA, 2.0 mg). A significance increase in lesion area was detected in control S.C. group and Oral GA (250 μg) groups compared to time 0 (Tuket test, P<0.05).

High-dose GA (2.0 mg) administered S.C. inhibited lesion area progression significantly (t-test, P=0.009) by 52% compared to control group III (FIG. 8).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for treating or preventing cardiovascular diseases and disorders in a subject in need thereof, comprising administering a pharmaceutical composition comprising a therapeutically effective amount of at least one copolymer, the copolymer selected from copolymer 1 and a copolymer 1-related heteropolymer wherein said copolymer comprises at least three amino acids each one selected from at least three of the following groups:
(a) lysine and arginine;
(b) glutamic acid and aspartic acid;
(c) alanine, glycine and valine;
(d) tyrosine, tryptophan and phenylalanine.

2. The method of claim 1, wherein the at least one copolymer is selected from the group consisting of a random copolymer, an ordered copolymer and an ordered peptide.

3. The method of claim 2, wherein said at least one copolymer contains four different amino acids each selected from one of groups (a) to (d).

4. The method of claim 1, wherein said at least one copolymer consists essentially of alanine, glutamic acid, lysine, and tyrosine, of net overall positive electrical charge and of a molecular weight of about 2,000 to about 40,000 daltons.

5. The method of claim 4 wherein the molecular weight of said at least one copolymer is of about 13,000 to about 18,000 daltons.

6. The method of claim 5, wherein the molecular weight of said at least one copolymer is about 15 kilodaltons.

7. The method of claim 4, wherein said at least one copolymer consists of alanine, glutamic acid, lysine, and tyrosine in the molar ratios of about 0.14 glutamic acid, about 0.43 alanine, about 0.10 tyrosine and about 0.33 lysine.

8. The method of claim 7, wherein said molar ratios are of 0.17 glutamic acid, 0.49 alanine, 0.10 tyrosine and 0.38 lysine.

9. The method of claim 7, wherein said molar ratios are of 0.19 glutamic acid, 0.6 alanine, 0.10 tyrosine and 0.4 lysine.

10. The method of claim 1, wherein said copolymer is glatiramer acetate.

11. The method of claim 1, wherein said at least one copolymer is a terpolymer containing three different amino acids each selected from groups (a) to (d).

12. The method of claim 11, wherein the terpolymer consists essentially of three different amino acids each selected from any one of groups (a), (c) and (d).

13. The method of claim 12, wherein said terpolymer consists essentially of tyrosine, alanine and lysine, in the molar ratio of from about 0.005 to about 0.25 tyrosine, from about 0.3 to about 0.6 alanine, and from about 0.1 to about 0.5 lysine.

14. The method of claim 13, wherein said terpolymer is YAK, having a molecular weight of 2000-40000 daltons.

15. The method of claim 11, wherein said terpolymer consists essentially of three different amino acids each selected from one of groups (a), (b) and (d).

16. The method of claim 15, wherein said terpolymer consists essentially of the amino acids glutamic acid, tyrosine, and lysine in the molar ratio of from about 0.005 to about 0.300 glutamic acid, from about 0.005 to about 0.250 tyrosine, and from about 0.3 to about 0.7 lysine.

17. The method of claim 16, wherein said terpolymer is YEK, having a molecular weight of about 2000-40000 Da.

18. The method of claim 11, wherein said terpolymer consists essentially of three different amino acids each selected from one of groups (b), (c) and (d).

19. The method of claim 18, wherein said terpolymer consists essentially of the amino acids tyrosine, glutamic acid, and alanine in the molar ratio of from about 0.005 to about 0.25 tyrosine, from about 0.005 to about 0.3 glutamic acid, and from about 0.005 to about 0.8 alanine.

20. The method of claim 19, wherein said terpolymer is YEA having a molecular weight of about 2000-40000 Da.

21. The method according to claim 1, wherein the amino acids comprising the at least one copolymer are selected from the group consisting of: L-amino acids, D-amino acids and a mixture of L- and D-amino acids.

22. The method of claim 2, wherein the ordered peptide is selected from SEQ ID NOS: 1-32.

23. The method of claim 1, wherein the at least one copolymer is a random copolymer comprising about 15 to about 100 amino acids.

24. The method of claim 1, further comprising administering the at least one copolymer in combination with at least one immunomodulating agent, the at least one immunomodulating agent being capable of attenuating the activity or presence of Th1 lymphocytes.

25. The method of claim 24, wherein the immunomodulating agent is an antibody selected from the group consisting of: α-IL-4, α-IL-12, α-IL-18, α-IFN-γ, γ-integrin and α-CD4⁺.

26. The method of claim 24, wherein said immunomodulating agent is capable of reducing the expression of molecules selected from the group consisting of IL-12, IL-18, integrin and IFN-γ.

27. The method of claim 26, wherein said immunomodulating agent is selected from the group consisting of antisense nucleotide sequence, sense nucleotide sequence, interfering RNA, ribozyme and aptamer.

28. The method of claim 24, wherein said at least one copolymer and said at least one immunomodulating agent are administered sequentially.

29. The method of claim 24, wherein said at least one copolymer and said at least one immunomodulating agent are administered substantially at the same time.

30. The method of claim 24, wherein said at least one copolymer and said at least one immunomodulating agent are co-administered in a single composition.

31. The method of claim 24, wherein said at least one copolymer and said at least one immunomodulating agent are administered in separate compositions.

32. The method of claim 1, wherein the cardiovascular disease and disorder is selected from the group consisting of arteriosclerosis, atherosclerosis and myocarditis.

33. The method of claim 1, wherein said pharmaceutical composition is formulated in a unit dosage form suitable for oral administration.

34. The method of claim 1, wherein said pharmaceutical composition is formulated in a unit dosage form suitable for parenteral injection.

35. The method of claim 1, wherein the therapeutically effective amount of the at least one copolymer ranges from about 1.0 mg to about 500.0 mg/day.

36. The method of claim 35, wherein the therapeutically effective amount of the at least one copolymer ranges from about 20.0 mg to about 100.0 mg/day.
